(19) United States
(12) Patent Application Publication
(10) Pub. No.: US 2024/0335828 A1
(43) Pub. Date: Oct. 10, 2024

(54) GAS-PHASE ALDEHYDE HYDROGENATION CATALYST, PREPARATION METHOD THEREOF AND APPLICATION THEREOF

(71) Applicant: PetroChina Company Limited, Beijing (CN)

(72) Inventors: Shurong NI, Beijing (CN); Xianjun WU, Beijing (CN); Gang WANG, Beijing (CN); Endong XIA, Beijing (CN); Famin SUN, Beijing (CN); Zidong WANG, Beijing (CN); Yu LIANG, Beijing (CN); Hongyu TIAN, Beijing (CN); Ruifeng LI, Beijing (CN)

(21) Appl. No.: 18/746,663

(22) Filed: Jun. 18, 2024

Related U.S. Application Data
(63) Continuation of application No. PCT/CN2022/139425, filed on Dec. 15, 2022.

(30) Foreign Application Priority Data
Dec. 29, 2021 (CN) 202111646159.7

(51) Int. Cl. B01J 37/04; B01J 21/04; B01J 23/80; B01J 37/08; C07C 29/141

(52) U.S. Cl. CPC B01J 37/04; B01J 21/04; B01J 23/80; B01J 37/08; C07C 29/141

(57) ABSTRACT

A gas-phase aldehyde hydrogenation catalyst, a preparation method thereof and an application thereof, where the preparation method includes: mixing an aluminum salt, a first zinc salt, silica sol, a first precipitant and water, and performing a first aging treatment to obtain a first mixed solution; mixing a copper salt, a second zinc salt, a zinc powder and water, and performing an ultrasonic treatment to obtain a second mixed solution; mixing the first mixed solution, the second mixed solution and a second precipitant, and performing a second aging treatment to obtain a third mixed solution; adding boric acid into the third mixed solution, and performing a third aging treatment to obtain a fourth mixed solution; filtering the fourth mixed solution, and sequentially drying and roasting the obtained solid product to obtain a catalyst precursor; mixing and molding the catalyst precursor with a graphite to obtain a gas-phase aldehyde hydrogenation catalyst.

The present application is a continuation of International Application No. PCT/CN2022/139425, filed on Dec. 15, 2022, which claims priority to Chinese Patent Application No. 202111646159.7, filed with China National Intellectual Property Administration on Dec. 29, 2021, entitled with “Gas-phase aldehyde hydrogenation catalyst, preparation method thereof and application thereof”. The disclosures of the aforementioned applications are hereby incorporated by reference in their entireties.

### TECHNICAL FIELD

The present application relates to the technical field of catalysts, and, in particular, to a gas-phase aldehyde hydrogenation catalyst, a preparation method thereof and an application thereof.

### BACKGROUND

The currently used gas-phase aldehyde hydrogenation catalysts are mainly copper-zinc (Cu—Zn) catalysts, which have low pollution, long life and high mechanical strength and other characteristics. In Cu—Zn catalysts, Cu acts as active component, and ZnO acts as carrier and additive. Generally, a Cu—Zn powder is prepared by a co-precipitation method, and then the powder obtained by the co-precipitation method is pressed and molded to obtain a Cu—Zn cylindrical catalyst.

For example, patent document CN105080549B discloses a catalyst for gas-phase hydrogenation of octenal to 2-ethylhexanol and a preparation method thereof, where the catalyst is prepared by a stepwise precipitation method, and then the catalyst is used to prepare octanol through octenal hydrogenation. Patent document CN100398202C discloses a catalyst for gas-phase hydrogenation of aldehyde and a preparation method thereof, where the catalyst is prepared by a continuous co-precipitation method using a mixed solution of nitrates of copper, zinc and aluminium, and the obtained catalyst is used for hydrogenation of butyraldehyde and octenal. Patent document CN108043411A discloses a catalyst for preparation of n-butyl alcohol through n-butyraldehyde hydrogenation and a preparation method thereof, where a synchronous precipitation method is used to enable copper and zinc to be precipitated at two different temperatures respectively so as to obtain two different pore structures and thus form a double-pore-based catalyst. The above catalysts are mainly used in the hydrogenation of butyraldehyde and octenal, but catalysts for hydrogenation of C9+ heavy aldehydes, especially catalysts for hydrogenation of 2-propyl-heptaldehyde, have been less studied.

Therefore, how to provide a preparation method of a catalyst that can be used for catalytic conversion of heavy aldehydes and improvement of conversion rate of the heavy aldehydes is an urgent technical problem to be solved in the field.

### SUMMARY

The present application provides a gas-phase aldehyde hydrogenation catalyst, a preparation method thereof and an application thereof. The catalyst obtained by the preparation method can be used for catalytic conversion of aldehyde hydrogenation, particularly for heavy aldehyde hydrogenation catalysis. Meanwhile, the catalyst also has high catalytic activity and other advantages, which can improve the conversion rate of aldehydes.

A first aspect of the present application provides a preparation method of a gas-phase aldehyde hydrogenation catalyst, including the following steps: mixing an aluminum salt, a first zinc salt, silica sol, a first precipitant and water, with system pH being controlled to be 7-8, and performing a first aging treatment to obtain a first mixed solution, where a molar ratio of the aluminum salt, the first zinc salt and the silica sol is 0.04-0.07:0.05-0.08:0.1-0.14; mixing a copper salt, a second zinc salt, a zinc powder and water, and performing an ultrasonic treatment to obtain a second mixed solution, where a molar ratio of the copper salt, the second zinc salt and the zinc powder is 0.2-5:0.1-5:0.1-5, and a molar ratio of the first zinc salt to the second zinc salt is 1:7-10; mixing the first mixed solution, the second mixed solution and a second precipitant, with system pH being controlled to be 7-8, and performing a second aging treatment to obtain a third mixed solution; adding boric acid into the third mixed solution, adjusting pH to 7-8, and performing a third aging treatment to obtain a fourth mixed solution; filtering the fourth mixed solution to obtain a solid product, and sequentially drying and roasting the obtained solid product to obtain a catalyst precursor; mixing and molding the catalyst precursor with a graphite to obtain the gas-phase aldehyde hydrogenation catalyst.

According to an embodiment of the present application, conditions of the ultrasonic treatment are: temperature of 70° C.-90° C., ultrasonic treatment power of 50 w-400 w and time of 5 s-600 s.

According to an embodiment of the present application, a temperature of the first aging treatment is 70° C.-90° C.; and/or a temperature of the second aging treatment is 70° C.-90° C.; and/or a temperature of the third aging treatment is 70° C.-90° C.

According to an embodiment of the present application, the first precipitant includes sodium carbonate; and/or the second precipitant includes sodium carbonate.

According to an embodiment of the present application, a volume ratio of the boric acid to the third mixed solution is 7-10:1500.

According to an embodiment of the present application, conditions of the roasting are: roasting temperature of 360° C.-400° C., and roasting time of 4 hours-5 hours.

According to an embodiment of the present application, a mass content of the graphite in the gas-phase aldehyde hydrogenation catalyst is 1%-3%.

According to an embodiment of the present application, after filtering the fourth mixed solution, the method further includes washing a filtered product with water of 40° C.-50° C. to obtain the solid product.

A second aspect of the present application provides a gas-phase aldehyde hydrogenation catalyst, which is obtained by the above preparation method.

A third aspect of the present application provides a gas-phase aldehyde hydrogenation method, including: subjecting an aldehyde-containing feedstock to a hydrogenation reaction in the presence of a catalyst to obtain a hydrogenation product; where the catalyst includes the above gas-phase aldehyde hydrogenation catalyst.

The embodiments of the present application have at least the following beneficial effects:

According to the preparation method of the gas-phase aldehyde hydrogenation catalyst provided by the present application, an aluminium salt, a first zinc salt, silica sol, a first precipitant and water are first mixed for aging and preliminary precipitation to obtain a first mixed solution; then a copper salt, a second zinc salt, a zinc powder and water are mixed to obtain a second mixed solution, in which copper ions are reduced into copper monomer by zinc monomer through an electrochemical replacement reaction; the first mixed solution and the second solution are mixed to introduce zinc-copper bimetal, so that a copper-zinc alloy with a double-electron effect is formed by an electronic interaction between copper and zinc; the copper-zinc alloy is enabled to exist on the surface of primary precipitation products, so that a surface electronic effect of the catalyst is improved, which is conducive to the hydrogenation reaction, thereby improving the catalytic performance of the catalyst; meanwhile, ultrasonic is adopted to assist the electrochemical replacement reaction, which can improve the dispersion of active metal in the catalyst and enhance the stability of the catalyst; a modifying additive such as boric acid or borate is introduced for continuous precipitation, which further improves the specific surface area and stability of the catalyst.

The catalyst prepared by the above method has a bimetallic site and the bimetallic site has a synergistic effect, which is conducive to improving the adsorption of the catalyst on heavy aldehydes, and the bimetallic site has high dispersibility, which can further improve the conversion efficiency of heavy aldehydes. Specifically, the catalyst provided by the present application can be used for hydrogenation catalysis of heavy aldehydes such as 2-propyl-heptenal, and can achieve a conversion rate of octenal of more than 99.98% and a conversion rate of 2-propyl-heptenal of more than 99.28%.

### DESCRIPTION OF EMBODIMENTS

The follow detailed description is merely illustrative of the principle and features of the present application, and the examples are provided for the purpose of explain the present application and are not intended to limit the scope of the present application. All other embodiments obtained by persons of ordinary skill in the art based on embodiments of the present application without creative effort shall fall within the protection scope of the present application.

A preparation method of a gas-phase aldehyde hydrogenation catalyst provided in the present application includes the following steps: mixing an aluminum salt, a first zinc salt, silica sol, a first precipitant and water, performing a first aging treatment, with system pH being controlled to be 7-8 to obtain a first mixed solution, where a molar ratio of the aluminum salt, the first zinc salt and the silica sol is 0.04-0.07:0.05-0.08:0.1-0.14; mixing a copper salt, a second zinc salt, a zinc powder and water, and performing an ultrasonic treatment to obtain a second mixed solution, where a molar ratio of the copper salt, the second zinc salt and the zinc powder is 0.2-5:0.1-5:0.1-5, and a molar ratio of the first zinc salt to the second zinc salt is 1:7-10; mixing the first mixed solution, the second mixed solution and a second precipitant, performing a second aging treatment with system pH being controlled to be 7-8 to obtain a third mixed solution; adding a boric acid into the third mixed solution, adjusting pH to 7-8, and performing a third aging treatment to obtain a fourth mixed solution; filtering the fourth mixed solution, and sequentially drying and roasting an obtained solid product to obtain a catalyst precursor; mixing and molding the catalyst precursor with a graphite to obtain the gas-phase aldehyde hydrogenation catalyst.

In the present application, the aluminum salt, the first zinc salt, the silica sol, the first precipitant and water are mixed, the system pH is controlled to be 7-8, and the first aging treatment is performed to obtain the first mixed solution. For the above operations, a specific process includes the following steps: mixing the aluminum salt, the first zinc salt, the silica sol and water first to form a mixed solution containing the aluminum salt, the first zinc salt and the silicon (aluminum-zinc-silicon mixed solution); and then adding the first precipitant into the mixed solution containing the aluminum salt, the first zinc salt and the silica sol, controlling the system pH to be 7-8, and performing the first aging treatment to obtain the first mixed solution. In a specific implementation process of the present application, the first precipitant is first mixed with water to form a solution containing the first precipitant, and then the solution containing the first precipitant is added to the above system for mixing. In the solution containing the first precipitant, a mass content of the first precipitant is not limited here, for example, 15%-30%, such as 15%, 18%, 20%, 25%, 30% or a range consisting of any two thereof, with system pH being controlled to be 7-8, such as 7, 7.2, 7.5, 7.8, 8 or a range consisting of any two thereof.

Generally, the aluminum salt is a salt containing aluminum ions, such as aluminum nitrate; the zinc salt is a salt containing zinc ions, such as zinc nitrate; and the silica sol contains silica dispersed in a solvent. The first mixed solution at least contains primary precipitation products of aluminum, zinc and silicon, and an amount of the primary precipitation products may be regulated by controlling amounts of the added substances. In some embodiments, a molar ratio of the aluminum salt, the first zinc salt and the silica sol is 0.04-0.07:0.05-0.08:0.1-0.14, where a molar ratio of the aluminum salt and the first zinc salt is calculated according to molar quantities of aluminium ions and of zinc ions therein, respectively, and a molar ratio of the silica sol is calculated according to molar quantities of silica.

Specifically, the first precipitant is an alkaline precipitant, and addition of the alkaline precipitant may enable aluminum, zinc and silicon in the system to be precipitated out in the form of precipitation by, and may also achieve the purpose of controlling the system pH to be 7-8. The alkaline precipitant includes sodium carbonate, potassium carbonate, sodium bicarbonate, potassium bicarbonate, sodium hydroxide, potassium hydroxide, ammonium carbonate, ammonium bicarbonate and ammonia. In some embodiments, the first precipitant is sodium carbonate.

In the present application, the first aging treatment is performed in a tank container, such as a neutralization tank. In a specific implementation process of the present application, an aluminum salt, a first zinc salt, a silica sol and water are first mixed to form a mixed solution containing the aluminum salt, the first zinc salt and the silica sol, and then the mixed solution and a solution containing a first precipitant are incorporated into the neutralization tank for the first aging treatment. In addition, the neutralization tank can be heated to control of the temperature within the tank. In some embodiments, a temperature of the first aging treatment is 70° C.-90° C., such as 70° C., 75° C., 80° C., 85° C., 90° C., or a range consisting of any two thereof, which is achieved by regulating the temperature of the neutralization tank.

In the present application, a copper salt, a second zinc salt, a zinc powder and water are mixed and then an ultrasonic treatment is performed to obtain a second mixed solution. The process specifically includes the following steps: mixing the copper salt, the second zinc salt and water first to form a copper-zinc mixed solution, adding the zinc powder into the copper-zinc mixed solution, stirring, and then performing the ultrasonic treatment to obtain a second mixed solution, where the zinc powder contains zinc monomer, and the copper-zinc mixed solution contains copper ions and zinc ions. Zinc powder is added into the copper-zinc mixed solution and then stirred, and in the process of the ultrasonic treatment, copper ions are reduced into copper monomer by zinc monomer through an electrochemical replacement reaction to generate a zinc-copper bimetal, and a copper-zinc alloy with a double-electron effect is formed by the electronic interactions between copper and zinc. The second mixed solution at least contains the copper-zinc alloy, copper ions unreacted and zinc ions unreacted. In addition, the dispersion of active metal can be promoted in the ultrasonic-assisted electrochemical replacement method.

In some embodiments, a molar ratio of the copper salt, the second zinc salt and the zinc powder is 0.2-5:0.1-5:0.1-5, where the copper salt is calculated according to mole quantities of copper ions and the second zinc salt is calculated according to mole quantities of zinc ions.

Generally, the zinc powder contains zinc monomer. In a specific process of the present application, the zinc powder meets the following conditions: zinc purity≥99% and particle size≤0.15 mm, which is more likely to promote the zinc powder and the copper ions in the solution to fully contact for the replacement reaction.

In some embodiments, conditions of the ultrasonic treatment are: temperature of 70° C.-90° C., such as 70° C., 75° C., 80° C., 85° C., 90° C. or a range consisting of any two thereof, ultrasonic treatment power of 50 w-400 w, such as 50 w, 100 w, 150 w, 200 w, 250 w, 300 w, 350 w, 400 w or a range consisting of any two thereof, and time of 5 s-600 s, such as 5 s, 10 s, 50 s, 60 s, 70 s, 100 s, 200 s, 300 s, 400 s, 500 s, 600 s or a range consisting of any two thereof.

In the present application, the first mixed solution, the second mixed solution and the second precipitant are mixed, the system pH is controlled to be 7-8, and the second aging treatment is performed to obtain the third mixed solution. For the above operations, a specific process includes the following steps: mixing the second precipitant with water first to form a solution containing the second precipitant, adding the second mixed solution and the solution containing the second precipitant into the first mixed solution, stirring, with system pH being controlled to be 7-8, and then performing a second aging treatment to obtain a third mixed solution.

Specifically, the second precipitant is an alkaline precipitant, and addition of the alkaline precipitant may precipitate the unreacted copper ions and zinc ions in the system in the form of precipitation, and may also achieve the purpose of controlling the system pH to be 7-8. The alkaline precipitant includes sodium carbonate, potassium carbonate, sodium bicarbonate, potassium bicarbonate, sodium hydroxide, potassium hydroxide, ammonium carbonate, ammonium bicarbonate and ammonia. In some embodiments, the first precipitant is sodium carbonate.

In some embodiments, a molar ratio of the first zinc salt to the second zinc salt is from 1:7-10, such as 1:7, 1:7.8, 1:8, 1:8.3, 1:9, 1:10, or a range consisting of any two thereof.

Specifically, the second aging treatment is performed in a tank container, such as a neutralization tank. In the specific implementation process of the present application, the first mixed solution is placed in the neutralization tank, and then the second mixed solution and the solution containing the second precipitant are incorporated into the neutralization tank for the second aging treatment. In addition, the neutralization tank can be heated to control the temperature within the tank. In some embodiments, a temperature of the second aging treatment is 70° C.-90° C., such as 70° C., 75° C., 80° C., 85° C., 90° C., or a range consisting of any two thereof, which is achieved by regulating the temperature of the neutralization tank. Through the second aging treatment, the copper-zinc alloy in the second mixed solution can exist on the surface of the primary precipitation products in the first mixed solution. For example, the copper-zinc alloy is coated on the surface of the primary precipitation products to form a coated copper-zinc alloy. The copper-zinc alloy existing on the surface of the primary precipitation products can improve the surface electronic effect of the catalyst, which is conducive to the hydrogenation reaction, thereby improving the catalytic performance of the catalyst. Meanwhile, ultrasonic is adopted to assist the electrochemical reaction, which can improve the dispersion of active metal in the catalyst and further enhance the stability of the catalyst.

In the present application, boric acid is added into the third mixed solution, the pH is adjusted to 7-8, a third aging treatment is performed to obtain a fourth mixed solution. In a specific implementation process of the present application, an aqueous boric acid solution is added into the third mixed solution, and after stirring, the pH is adjusted to 7-8, and then the third aging treatment is performed to obtain the fourth mixed solution. In the above process, the specific surface area and stability of the catalyst are further improved by introducing a modifying additive such as boron for continuous precipitation.

Specifically, the process of adding the aqueous boric acid solution to the third mixed solution, stirring, and adjusting the pH to 7-8 includes using a third precipitant to adjust the pH. The third precipitant is an alkaline precipitant, and by adding the alkaline precipitant, boron in the system may be precipitated out in the form of precipitation, and the purpose of controlling a system pH to 7-8 may also be achieved. The alkaline precipitant includes sodium carbonate, potassium carbonate, sodium bicarbonate, potassium bicarbonate, sodium hydroxide, potassium hydroxide, ammonium carbonate, ammonium bicarbonate and ammonia. In some embodiments, the third precipitant is sodium carbonate.

Specifically, the third aging treatment is performed in a tank container, such as a neutralization tank. In a specific implementation process of the present application, the third mixed solution is first placed in the neutralization tank, and then the aqueous boric acid solution is introduced into the neutralization tank for the third aging treatment. In addition, the neutralization tank can be heated to control the temperature within the tank. In some embodiments, a temperature of the third aging treatment is 70° C.-90° C., such as 70° C., 75° C., 80° C., 85° C., 90° C., or a range consisting of any two thereof, which is achieved by regulating the temperature of the neutralization tank.

In some embodiments, a volume ratio of the aqueous boric acid solution to the third mixed solution is 7-10:1500, such as 7:1500, 7.5:1500, 8.5:1500, 9.5:1500, or a range consisting of any two thereof. The volume of the third mixed solution is approximately equal to the sum of the volumes of the first mixed solution and the second mixed solution. In some embodiments, a mass content of boric acid in the aqueous boric acid solution is 10%-20%, such as 10%, 11%, 12%, 13%, 14%, 15%, or a range consisting of any two thereof.

In the present application, the fourth mixed solution at least contains a primary precipitation products of copper, zinc and silicon, and final precipitation products such as copper-zinc alloy, copper precipitate and boron precipitate existing on the surface of the primary precipitation products. The fourth mixed solution is filtered to obtain a filtered product containing the final precipitation products.

In some embodiments, after filtering the fourth mixed solution, the method further includes washing the filtered product with deionized water of 40° C.-50° C. to obtain a solid product. The temperature of the deionized water is 40° C.-50° C., such as 40° C., 42° C., 43° C., 45° C., 48° C., 50° C., or a range consisting of any two thereof. The washed solid product is then sequentially dried and roasted to obtain a catalyst precursor, and a purpose of drying is to remove the redundant moisture in the solid product.

In some embodiments, conditions of the roasting treatment is: roasting temperature of 360° C.-400° C., such as 360° C., 370° C., 380° C., 390° C., 400° C., or a range consisting of any two thereof, and roasting time of 4 hours-5 hours, such as 4 hours, 4.2 hours, 4.3 hours, 4.4 hours, 4.5 hours, 4.8 hours, 5 hours, or a range consisting of any two thereof.

In the present application, the catalyst precursor obtained after roasting treatment and graphite are mixed and molded to obtain the gas-phase aldehyde hydrogenation catalyst, and a specific process includes the following steps: adding the graphite into the catalyst precursor, uniformly mixing, and then press moulding to obtain the gas-phase aldehyde hydrogenation catalyst. In some embodiments, a mass content of the graphite in the gas-phase aldehyde hydrogenation catalyst is 1%-3%, such as 1%, 1.2%, 1.5%, 1.8%, 2%, 2.5%, 3%, or a range consisting of any two thereof.

The gas-phase aldehyde hydrogenation catalyst provided by the present application is prepared by the above preparation method of the gas-phase aldehyde hydrogenation catalyst. Generally, the raw materials added in the preparation process can be fully converted into components of the gas-phase aldehyde hydrogenation catalyst. In some embodiments, compositions of the gas-phase aldehyde hydrogenation catalyst include, by mass: 20%-40% of copper oxide, such as 20%, 25%, 30%, 35%, 40% or a range consisting of any two thereof; 20%-40% of copper, such as 10%, 12%, 15%, 18%, 20% or a range consisting of any two thereof; 45%-70% of zinc oxide such as 45%, 50%, 55%, 60%, 70% or a range consisting of any two thereof; 5%-20% of zinc, such as 5%, 10%, 15%, 20% or a range consisting of any two thereof; 0.1%-6% of alumina, such as 0.1%, 0.5%, 1%, 2%, 4%, 5%, 6% or a range consisting of any two thereof; 1%-3% of graphite, such as 1%, 1.2%, 1.5%, 1.8%, 2%, 2.5%, 3% or a range consisting of any two thereof; 0.1%-5% of boron oxide, such as 0.1%, 0.5%, 1%, 2%, 4%, 5% or a range consisting of any two thereof; and 0.1% to 10% of silicon oxide, such as 0.1%, 0.5%, 1%, 2%, 4%, 5%, 6%, 10% or a range consisting of any two thereof.

The gas-phase aldehyde hydrogenation method provided in the present application includes: subjecting an aldehyde-containing feedstock to a hydrogenation reaction in the presence of a catalyst to obtain a hydrogenation product, where the catalyst includes the above gas-phase aldehyde hydrogenation catalyst. A specific process includes the following steps: feeding the above catalyst into a hydrogenation device, introducing a reaction gas into the hydrogenation device, and performing a hydrogenation reaction to obtain the hydrogenation product, and introducing nitrogen into the hydrogenation device to cool down after the hydrogenation reaction is finished. Where the feeding temperature is 135° C.-160° C., the reaction gas includes hydrogen and an aldehyde compound, where the aldehyde compound is in a gaseous state and a volume ratio of the hydrogen to the aldehyde compound is 7000-8000:1. The aldehyde compound includes at least one of octenal and 2-propyl-2-heptenal. Conditions of the hydrogenation reaction is: temperature of 205° C., reaction space velocity of 0.35 h⁻¹, reaction pressure of 0.44 MPa-0.55 MPa and reaction time of 4 hours-6 hours. The reaction space velocity refers to a volume space velocity of the aldehyde compound.

To make the objectives, technical solutions, and advantages of the present application clearer, the technical solutions of the present application will be described clearly and comprehensively below with reference to the examples of the present application. Apparently, the described examples are merely some rather than all embodiments of the present application. All other examples obtained by persons of ordinary skill in the art based on examples of the present application without creative effort shall fall within the protection scope of the present application.

### Example 1

(1) 10 g of aluminium nitrate, 10 g of zinc nitrate, 6 g of silica sol and water were mixed to obtain 200 mL of aluminium-zinc-silicon mixed solution, the aluminium-zinc-silicon mixed solution and a sodium carbonate solution are incorporated into a neutralization tank for stirring, with system pH being controlled to be 7.5, and then a first aging treatment was performed to obtain a first mixed solution, where a temperature of the first aging treatment was 80° C., and a mass fraction of sodium carbonate in the sodium carbonate solution was 20%;
(2) 117 g of copper nitrate, 100 g of zinc nitrate and water were mixed to form 1300 mL of copper-zinc mixed solution, 35 g of zinc powder was added into the solution for stirring, and then an ultrasonic treatment was performed to obtain a second mixed solution, where the zinc powder had a Zn purity of ≥99% and a particle size of ≤0.15 mm, and the ultrasonic treatment was performed for 60 s at a temperature of 80° C. and an ultrasonic power of 200 w;
(3) The second mixed solution and a sodium carbonate solution are incorporated into the tank containing the first mixed solution in step (1), with system pH being controlled to be 7.5, and then a second aging treatment was performed to obtain a third mixed solution, where a temperature of the second aging treatment was 80° C., and a mass fraction of sodium carbonate in the sodium carbonate solution was 20%;
(4) 8.5 mL of boric acid solution was added into the tank containing the third mixed solution, a sodium carbonate solution was added for adjusting pH to 7.5, and then a third aging treatment was performed to obtain a fourth mixed solution, where a mass fraction of sodium carbonate in the sodium carbonate solution was 20%;
(5) The fourth mixed solution was filtered, and then the filtered product was washed with deionized water of 40° C. to obtain a solid product, and the obtained solid product was dried and then roasted at 380° C. for 4 hours to obtain a catalyst precursor;
(6) Graphite was added into the catalyst precursor for mixing uniformly, and press moulding was then performed to obtain a gas-phase aldehyde hydrogenation catalyst CA-1, where a mass content of graphite in the gas-phase aldehyde hydrogenation catalyst was 2%.

### Example 2

(1) 15 g of aluminium nitrate, 14 g of zinc nitrate, 6 g of silica sol and water were mixed to obtain 200 mL of aluminium-zinc-silicon mixed solution, the aluminium-zinc-silicon mixed solution and a sodium carbonate solution are incorporated into a neutralization tank for stirring, with system pH being controlled to be 7.5, and then a first aging treatment was performed to obtain a first mixed solution; where a temperature of the first aging treatment was 80° C., and a mass fraction of sodium carbonate in the sodium carbonate solution was 20%;
(2) 125 g of copper nitrate, 110 g of zinc nitrate and water were mixed to form 1300 mL of copper-zinc mixed solution, 40 g of zinc powder was added into the solution for stirring, and then an ultrasonic treatment was performed to obtain a second mixed solution, where the zinc powder had a Zn purity of ≥99% and a particle size of ≤0.15 mm, and the ultrasonic treatment was performed for 70 s at a temperature of 80° C. and an ultrasonic power of 150 w;
(3) The second mixed solution and a sodium carbonate solution are incorporated into the tank containing the first mixed solution in step (1), with system pH being controlled to be 7.5, and then a second aging treatment was performed to obtain a third mixed solution, where a temperature of the second aging treatment was 80° C. and a mass fraction of sodium carbonate in the sodium carbonate solution was 20%;
(4) 9.5 mL of boric acid solution was added into the tank containing the third mixed solution, a sodium carbonate solution was added for adjusting pH to 7.5, and then a third aging treatment was performed to obtain a fourth mixed solution, where a mass fraction of sodium carbonate in the sodium carbonate solution was 10%;
(5) The fourth mixed solution was filtered, and then the filtered product was washed with deionized water of 40° C. to obtain a solid product, and the obtained solid product was dried and then roasted at 380° C. for 4 hours to obtain a catalyst precursor;
(6) Graphite was added into the catalyst precursor for mixing uniformly, and press moulding was then performed to obtain a gas-phase aldehyde hydrogenation catalyst CA-2, where a mass content of graphite in the gas-phase aldehyde hydrogenation catalyst was 2%.

### Example 3

(1) 13 g of aluminium nitrate, 15 g of zinc nitrate, 8 g of silica sol and water were mixed to obtain 200 mL of aluminium-zinc-silicon mixed solution, the aluminium-zinc-silicon mixed solution and a sodium carbonate solution are incorporated into a neutralization tank for stirring, with system pH being controlled to be 7.5, and then a first aging treatment was performed to obtain a first mixed solution, where a temperature of the first aging treatment was 80° C., and a mass fraction of sodium carbonate in the sodium carbonate solution was 20%;
(2) 130 g of copper nitrate, 125 g of zinc nitrate and water were mixed to form 1300 mL of copper-zinc mixed solution, 42 g of zinc powder was added into the solution for stirring, and then an ultrasonic treatment was performed to obtain a second mixed solution, where the zinc powder had a Zn purity of ≥99% and a particle size of ≤0.15 mm, and the ultrasonic treatment was performed for 70 s at a temperature of 80° C. and an ultrasonic power of 200 w;
(3) The second mixed solution and a sodium carbonate solution are incorporated into the tank containing the first mixed solution in step (1), with system pH being controlled to be 7.5, and then a second aging treatment was performed to obtain a third mixed solution, where a temperature of the second aging treatment was 80° C. and a mass fraction of sodium carbonate in the sodium carbonate solution was 20%;
(4) 8.5 mL of boric acid solution was added into the tank containing the third mixed solution, a sodium carbonate solution was added for adjusting pH to 7.5, and then a third aging treatment was performed to obtain a fourth mixed solution, where a mass fraction of sodium carbonate in the sodium carbonate solution was 10%;
(5) The fourth mixed solution was filtered, and then the filtered product was washed with deionized water of 40° C. to obtain a solid product, and the obtained solid product was dried and then roasted at 380° C. for 4 hours to obtain a catalyst precursor;
(6) Graphite was added into the catalyst precursor for mixing uniformly, and press moulding was then performed to obtain a gas-phase aldehyde hydrogenation catalyst CA-3, where a mass content of graphite in the gas-phase aldehyde hydrogenation catalyst was 2%.

### Example 4

(1) 13 g of aluminium nitrate, 30 g of zinc nitrate, 15 g of silica sol and water were mixed to obtain 200 mL of aluminium-zinc-silicon mixed solution, the aluminium-zinc-silicon mixed solution and a sodium carbonate solution are incorporated into a neutralization tank for stirring, with system pH being controlled to be 7.5, and then a first aging treatment was performed to obtain a first mixed solution, where a temperature of the first aging treatment was 80° C., and a mass fraction of sodium carbonate in the sodium carbonate solution was 20%;
(2) 250 g of copper nitrate, 125 g of zinc nitrate and water were mixed to form 1300 mL of copper-zinc mixed solution, 75 g of zinc powder was added into the solution for stirring, and then an ultrasonic treatment was performed to obtain a second mixed solution, where the zinc powder had a Zn purity of ≥99% and a particle size of ≤0.15 mm, and the ultrasonic treatment was performed for 70 s at a temperature of 80° C. and an ultrasonic power of 200 w;
(3) The second mixed solution and a sodium carbonate solution are incorporated into the tank containing the first mixed solution in step (1), with system pH being controlled to be 7.5, and then a second aging treatment was performed to obtain a third mixed solution, where a temperature of the second aging treatment was 80° C. and a mass fraction of sodium carbonate in the sodium carbonate solution was 20%;
(4) 14 mL of boric acid solution was added into the tank containing the third mixed solution, a sodium carbonate solution was added for adjusting pH to 7.5, and then a third aging treatment was performed to obtain a fourth mixed solution, where a mass fraction of sodium carbonate in the sodium carbonate solution was 10%;
(5) The fourth mixed solution was filtered, and then the filtered product was washed with deionized water of 40° C. to obtain a solid product, and the obtained solid product was dried and then roasted at 380° C. for 4 hours to obtain a catalyst precursor;
(6) Graphite was added into the catalyst precursor for mixing uniformly, and press moulding was then performed to obtain a gas-phase aldehyde hydrogenation catalyst CA-4, where a mass content of graphite in the gas-phase aldehyde hydrogenation catalyst was 2%.

### Example 5

(1) 30 g of aluminium nitrate, 16 g of zinc nitrate, 14 g of silica sol and water were mixed to obtain 200 mL of aluminium-zinc-silicon mixed solution, the aluminium-zinc-silicon mixed solution and a sodium carbonate solution are incorporated into a neutralization tank for stirring, with system pH being controlled to be 7.5, and then a first aging treatment was performed to obtain a first mixed solution, where a temperature of the first aging treatment was 80° C., and a mass fraction of sodium carbonate in the sodium carbonate solution was 20%;
(2) 130 g of copper nitrate, 300 g of zinc nitrate and water were mixed to form 1300 mL of copper-zinc mixed solution, 48 g of zinc powder was added into the solution for stirring, and then an ultrasonic treatment was performed to obtain a second mixed solution, where the zinc powder had a Zn purity of ≥99% and a particle size of ≤0.15 mm, and the ultrasonic treatment was performed for 70 s at a temperature of 80° C. and an ultrasonic power of 200 w;
(3) The second mixed solution and a sodium carbonate solution are incorporated into the tank containing the first mixed solution in step (1), with system pH being controlled to be 7.5, and then a second aging treatment was performed to obtain a third mixed solution, where a temperature of the second aging treatment was 80° C. and a mass fraction of sodium carbonate in the sodium carbonate solution was 20%;
(4) 16 mL of boric acid solution was added into the tank containing the third mixed solution, a sodium carbonate solution was added for adjusting pH to 7.5, and then a third aging treatment was performed to obtain a fourth mixed solution, where a mass fraction of sodium carbonate in the sodium carbonate solution was 10%;
(5) The fourth mixed solution was filtered, and then the filtered product was washed with deionized water of 40° C. to obtain a solid product, and the obtained solid product was dried and then roasted at 380° C. for 4 hours to obtain a catalyst precursor;
(6) Graphite was added into the catalyst precursor for mixing uniformly, and press moulding was then performed to obtain a gas-phase aldehyde hydrogenation catalyst CA-5, where a mass content of graphite in the gas-phase aldehyde hydrogenation catalyst was 2%.

### Example 6

(1) 14 g of aluminium nitrate, 16 g of zinc nitrate, 14 g of silica sol and water were mixed to obtain 200 mL of aluminium-zinc-silicon mixed solution, the aluminium-zinc-silicon mixed solution and a sodium carbonate solution are incorporated into a neutralization tank for stirring, with system pH being controlled to be 7.5, and then a first aging treatment was performed to obtain a first mixed solution, where a temperature of the first aging treatment was 80° C., and a mass fraction of sodium carbonate in the sodium carbonate solution was 20%;
(2) 390 g of copper nitrate, 50 g of zinc nitrate and water were mixed to form 1300 mL of copper-zinc mixed solution, 60 g of zinc powder was added into the solution for stirring, and then an ultrasonic treatment was performed to obtain a second mixed solution, where the zinc powder had a Zn purity of ≥99% and a particle size of ≤0.15 mm, and the ultrasonic treatment was performed for 70 s at a temperature of 80° C. and an ultrasonic power of 200 w;
(3) The second mixed solution and a sodium carbonate solution are incorporated into the tank containing the first mixed solution in step (1), with system pH being controlled to be 7.5, and then a second aging treatment was performed to obtain a third mixed solution, where a temperature of the second aging treatment was 80° C. and a mass fraction of sodium carbonate in the sodium carbonate solution was 20%;
(4) 16 mL of boric acid solution was added into the tank containing the third mixed solution, a sodium carbonate solution was added for adjusting pH to 7.5, and then a third aging treatment was performed to obtain a fourth mixed solution, where a mass fraction of sodium carbonate in the sodium carbonate solution was 10%;
(5) The fourth mixed solution was filtered, and then the filtered product was washed with deionized water of 40° C. to obtain a solid product, and the obtained solid product was dried and then roasted at 380° C. for 4 hours to obtain a catalyst precursor;
(6) Graphite was added into the catalyst precursor for mixing uniformly, and press moulding was then performed to obtain a gas-phase aldehyde hydrogenation catalyst CA-6, where a mass content of graphite in the gas-phase aldehyde hydrogenation catalyst was 2%.

### Comparative Example 1

(1) 10 g of aluminium nitrate, 10 g of zinc nitrate, 7 g of silica sol and water were mixed to obtain 200 mL of aluminium-zinc-silicon mixed solution, the aluminium-zinc-silicon mixed solution and a sodium carbonate solution are incorporated into a neutralization tank for stirring, with system pH being controlled to be 7.5, and then a first aging treatment was performed to obtain a first mixed solution; where a temperature of the first aging treatment was 80° C., and a mass fraction of sodium carbonate in the sodium carbonate solution was 20%;
(2) 117 g of copper nitrate, 210 g of zinc nitrate and water were mixed to form 1300 mL of copper-zinc mixed solution, the solution after being stirred and a sodium carbonate solution are incorporated into the tank containing the first mixed solution in step (1), with system pH being controlled to be 7.5, and then a second aging treatment was performed to obtain a second mixed solution; where a temperature of the second aging treatment was 80° C. and a mass fraction of sodium carbonate in the sodium carbonate solution was 20%;
(3) The second mixed solution was filtered, and then the filtered product was washed with deionized water of 45° C. to obtain a solid product, and the obtained solid product was dried and then roasted at 380° C. for 4 hours to obtain a catalyst precursor;
(4) Graphite was added into the catalyst precursor for mixing uniformly, and press moulding was then performed to obtain a gas-phase aldehyde hydrogenation catalyst CB-1, where a mass content of graphite in the gas-phase aldehyde hydrogenation catalyst was 2%.

### Comparative Example 2

A copper nitrate solution, a zinc nitrate solution and an aluminum nitrate solution were mixed to obtain a mixed solution A, where the copper nitrate solution had a concentration of 1 mol/L and a volume of 1000 mL, and the zinc nitrate solution had a concentration of 1 mol/L and a volume of 1773 mL; the aluminum nitrate solution had a concentration of 1 mol/L and a volume of 100 mL;

55 g of silica sol was added dropwise into a sodium carbonate solution at a temperature of 60° C. for aging to obtain a mixed solution B, where the aging was performed at a temperature of 65° C. for 20 minutes and where a concentration of the sodium carbonate solution is 1 mol/L;

The mixed solution A was added into the mixed solution B at a constant speed and reacted for 30 minutes, then aged with stirring at 70° C. for 30 minutes, and then a catalyst precursor was obtained by washing, press-filtering, drying and roasting, where conditions of the roasting was: temperature of 320° C., and time of 2 h;

0.5 g of a modifying additive was added into the catalyst precursor, and a catalyst sample CB-2 was obtained by press moulding.

### Comparative Example 3

6 g of zinc nitrate, 6 g of aluminum nitrate and water were mixed to obtain 100 mL of zinc-aluminum mixed solution; the zinc-aluminum mixed solution and a sodium carbonate solution are incorporated into a neutralization tank for aging, with pH being adjusted to 7.5, where a temperature of the neutralization tank was controlled to be 80° C., and a mass fraction of the sodium carbonate solution was 15.7%;

117 g of copper nitrate, 171 g of zinc nitrate and water were mixed to form 1200 mL of copper-zinc mixed solution, and then the copper-zinc mixed solution and a sodium carbonate solution are incorporated into the neutralization tank and aged to obtain a mixed solution, where a mass fraction of the sodium carbonate solution was 15.7%;

The mixed solution was filtered, the filtered product was washed with deionized water of 40° C., and then the filtered product was dried and roasted sequentially to obtain a catalyst precursor, where conditions of the roasting was: temperature of 380° C., and time of 4 hours;

Graphite was added into the catalyst precursor for mixing uniformly, and press moulding was then performed to obtain a catalyst sample CB-3.

The physical property indexes of the catalysts prepared in the examples and the comparative examples are shown in Table 1.

TABLE 1

| | Catalyst | | | | | | | | |
|---|---|---|---|---|---|---|---|---|---|
| | CA-1 | CA-2 | CA-3 | CA-4 | CA-5 | CA-6 | CB-1 | CB-2 | CB-3 |
| Pore volume | 0.28 | 0.27 | 0.26 | 0.21 | 0.29 | 0.22 | 0.28 | 0.26 | 0.23 |
| (mL/g) | | | | | | | | | |
| Specific | 66.7 | 66.5 | 66.6 | 66.6 | 67.6 | 63.6 | 66.5 | 65.6 | 69.4 |
| surface area | | | | | | | | | |
| (m²/g) | | | | | | | | | |
| Average pore | 15.1 | 14.9 | 15.0 | 12.8 | 15.7 | 11.9 | 11.9 | 10.6 | 9.6 |
| size (nm) | | | | | | | | | |

In the present application, the catalysts in the examples and the comparative examples were used to perform catalytic conversions of octenal and 2-propyl-2-heptenal, respectively, and specific processes were performed in a 200 ml fixed bed hydrogenation device, where reaction conditions for catalytic conversion of octenal were as follows: feedstock was octenal, feed temperature was 160° C., volume ratio of hydrogen to octenal was 8000:1, reaction space velocity was 0.35 h⁻¹, reaction pressure was 0.45 MPa; hydrogenation temperature was 205° C., time was 6 h, and nitrogen was used to cool down at the end of reduction;

The reaction conditions for catalytic conversion of 2-propyl-2-heptenal were as follows: feedstock was 2-propyl-2-heptenal, feed temperature was 135° C., volume ratio of hydrogen to 2-propyl-2-heptenal was 8000:1, reaction space velocity was 0.35 h⁻, and reaction pressure was 0.55 MPa.

The performance results of catalytic hydrogenation of octenal using the catalysts of the examples and the comparative examples were shown in Table 2.

TABLE 2

| | Item | | | | | | | | |
|---|---|---|---|---|---|---|---|---|---|
| | CA-1 | CA-2 | CA-3 | CA-4 | CA-5 | CA-6 | CB-1 | CB-2 | CB-3 |
| Conversion rate | 99.99 | 99.98 | 99.98 | 98.78 | 97.95 | 98.38 | 97.87 | 97.77 | 97.80 |
| of octenal, wt % | | | | | | | | | |
| Octanol | 99.85 | 99.83 | 99.84 | 99.34 | 98.76 | 99.15 | 98.60 | 98.62 | 98.72 |
| selectivity, % | | | | | | | | | |

The performance results of catalytic hydrogenation of 2-propyl-2-heptenal using the catalysts of the examples and the comparative examples were shown in Table 3.

TABLE 3

| | Item | | | | | | | | |
|---|---|---|---|---|---|---|---|---|---|
| | CA-1 | CA-2 | CA-3 | CA-4 | CA-5 | CA-6 | CB-1 | CB-2 | CB-3 |
| Conversion rate of 2- | 99.39 | 99.28 | 99.30 | 98.31 | 98.01 | 98.17 | 98.07 | 97.92 | 98.04 |
| propyl-2-heptenal, wt % | | | | | | | | | |
| 2-propyl-heptanol | 96.81 | 96.75 | 96.72 | 95.61 | 95.22 | 95.33 | 95.32 | 95.29 | 95.37 |
| selectivity, % | | | | | | | | | |

It can be seen from Table 1 that the catalysts in the examples and the comparative examples have basically the same pore volume and specific surface area, but the catalysts in the examples have significantly higher pore sizes than those in the comparative examples. Generally, a large pore size is conducive to diffusion of reactant in the interior of a catalyst. When the pore size of the catalyst is small, a heavy component with large molecular weight in the reactants is difficult to diffuse, which is not conducive to hydrogenation.

It can be seen from Tables 2 and 3 that the catalysts prepared by the preparation method of the present application have higher catalytic activity, which is embodied in that the catalytic conversion of octenal and 2-propyl-2-heptenal when using the catalysts of the present application showed higher conversion rate and selectivity than when using the catalysts of the comparative examples.

To sum up, the catalysts provided by the present application have the advantage of high catalytic activity, and can be used not only for hydrogenation of octenal to produce octanol, but also for catalytic conversion of C9+ heavy aldehydes, for example, for hydrogenation of 2-propyl-2-heptenal to produce 2-propyl-heptanol, thereby improving the yield of catalytic products.

The foregoing is a detailed description of the specific examples of the present application and experimental verification thereof. It should be understood that many modifications and variations can be made according to the concepts of the present application by ordinary skill in the art without creative effort. Therefore, all technical solutions that can be obtained by those skilled in the art through logical analysis, reasoning or limited experiments according to the concepts of the present application on the basis of the prior art shall fall within the protection scope defined in the claims.

What is claimed is:

1. A preparation method of a gas-phase aldehyde hydrogenation catalyst, comprising the following steps:
mixing an aluminum salt, a first zinc salt, silica sol, a first precipitant and water, with system pH being controlled to be 7-8, and performing a first aging treatment to obtain a first mixed solution, wherein a molar ratio of the aluminum salt, the first zinc salt and the silica sol is 0.04-0.07:0.05-0.08:0.1-0.14;
mixing a copper salt, a second zinc salt, a zinc powder and water, and performing an ultrasonic treatment to obtain a second mixed solution, wherein a molar ratio of the copper salt, the second zinc salt and the zinc powder is 0.2-5:0.1-5:0.1-5, and a molar ratio of the first zinc salt to the second zinc salt is 1:7-10;
mixing the first mixed solution, the second mixed solution and a second precipitant, with system pH being controlled to be 7-8, and performing a second aging treatment to obtain a third mixed solution;
adding boric acid into the third mixed solution, adjusting pH to 7-8, and performing a third aging treatment to obtain a fourth mixed solution;
filtering the fourth mixed solution, and sequentially drying and roasting an obtained solid product to obtain a catalyst precursor; and
mixing and molding the catalyst precursor with a graphite to obtain a gas-phase aldehyde hydrogenation catalyst.

2. The preparation method according to claim 1, wherein conditions of the ultrasonic treatment is: temperature of 70° C.-90° C., ultrasonic treatment power of 50 w-400 w and time of 5 s-600 s.

3. The preparation method according to claim 1, wherein a temperature of the first aging treatment is 70° C.-90° C.

4. The preparation method according to claim 1, wherein a temperature of the second aging treatment is 70° C.-90° C.

5. The preparation method according to claim 3, wherein a temperature of the second aging treatment is 70° C.-90° C.

6. The preparation method according to claim 1, wherein a temperature of the third aging treatment is 70° C.-90° C.

7. The preparation method according to claim 3, wherein a temperature of the third aging treatment is 70° C.-90° C.

8. The preparation method according to claim 1, wherein the first precipitant comprises sodium carbonate.

9. The preparation method according to claim 1, wherein the second precipitant comprises sodium carbonate.

10. The preparation method according to claim 8, wherein the second precipitant comprises sodium carbonate.

11. The preparation method according to claim 1, wherein a volume ratio of the boric acid to the third mixed solution is 7-10:1500.

12. The preparation method according to claim 1, wherein conditions of the roasting is: roasting temperature of 360° C.-400° C., and roasting time of 4-5 hours.

13. The preparation method according to claim 1, wherein a mass content of the graphite in the gas-phase aldehyde hydrogenation catalyst is 1%-3%.

14. The preparation method according to claim 1, wherein after filtering the fourth mixed solution, the method further comprises washing a filtered product with water of 40° C.-50° C. to obtain the solid product.

15. A gas-phase aldehyde hydrogenation catalyst, which is obtained by the preparation method according to claim 1.

16. The gas-phase aldehyde hydrogenation catalyst according to claim 15, wherein conditions of the ultrasonic treatment is: temperature of 70° C.-90° C., ultrasonic treatment power of 50 w-400 w and time of 5 s-600 s.

17. The gas-phase aldehyde hydrogenation catalyst according to claim 15, wherein a temperature of the first aging treatment is 70° C.-90° C.

18. The gas-phase aldehyde hydrogenation catalyst according to claim 15, wherein a temperature of the second aging treatment is 70° C.-90° C.

19. The gas-phase aldehyde hydrogenation catalyst according to claim 15, a temperature of the third aging treatment is 70° C.-90° C.

20. A gas-phase aldehyde hydrogenation method, comprising: subjecting an aldehyde-containing feedstock to a hydrogenation reaction in the presence of a catalyst to obtain a hydrogenation product, wherein the catalyst comprises the gas-phase aldehyde hydrogenation catalyst according to claim 15.
